(19) United States
(12) Patent Application Publication
(10) Pub. No.: US 2022/0134209 A1
(43) Pub. Date: May 5, 2022

(54) Fitness Device With a Stand

(71) Applicant: etone Motion Analysis GmbH, Berlin (DE)

(72) Inventors: Calin-Laurentiu Popescu, Berlin (DE); Valerie Bures-Bönström, Berlin (DE)

(21) Appl. No.: 17/086,708

(22) Filed: Nov. 2, 2020

(51) Int. Cl. A63B 71/06; G02B 7/182; G02B 27/14; G16H 40/63; G16H 20/30

(52) U.S. Cl. CPC A63B 71/0622; G02B 7/182; G06F 3/147; G16H 40/63; G16H 20/30; G02B 27/144

(57) ABSTRACT

A fitness device includes a frame assembly defining a front side and a back side and a frame structure which has a bottom part for placing the fitness device on a floor, at least one display fixed to the frame structure for displaying exercise to a user facing the front side, and a stand including an attachment portion attached to the back side of the frame assembly above the bottom part of the frame structure and a base portion to be placed on the floor.

### BACKGROUND OF THE INVENTION

### Field of the Invention

The present solution relates to a fitness device comprising at least one display for displaying exercise content to user facing a front side of the fitness device.

### Description of Related Art

Fitness devices which are to be used in gyms or at home and which include at least one display for displaying exercise content to a user become more and more popular. The exercise content may be a video retrieved from a local memory of the fitness device or from a memory of a computing device connected to the fitness device via a wired or wireless network. Alternatively or additionally, exercise content may be provided as a stream, for example via the Internet. Based on the exercise content the user is instructed how to perform different fitness exercises. In order to provide a new and motivating experience to the user and providing the user with sufficient details on how to carry out specific exercise properly, the at least one display of the fitness device may be rather large and held by a frame structure of the fitness device having more than 1 metre in length and more than 40 cm in width.

In this context fitness devices are also known which include a semi-reflective mirror arranged in front of the at least one display. Such a mirror may reflect incident light from a user facing a front surface of the mirror to present a reflected image of the user. Further, the mirror may transmit incident light from a rear surface of the mirror so that exercise content displayed via the at least one the display is superimposed on at least a portion of the reflected image of the user. Thereby, the user may see his/her reflected image in combination with an image of a trainer or avatar in the exercise content so that the user may easily notice whether a specific exercise is performed properly or not.

In addition, or as an alternative to a mirror, the fitness device may be equipped with a camera configured to capture the user while exercise content is displayed via the at least one display in order to provide electronic feedback on how well the user is the following the instructions of the exercise content and performs an exercise displayed to the user. The user may also receive visual and/or acoustic feedback on her/his current training.

In particular with respect to fitness devices used as stand-alone devices at a gym or at home it is crucial that these device may be easily and securely placed. Whereas hanging and thus rigidly fixing a fitness device at a wall might generally be an option, most customers prefer simply placing fitness devices on a floor to avoid cumbersome installations. It is thus an object of the present solution to provide for a fitness device which may be securely placed on a floor.

### SUMMARY OF THE INVENTION

According to a first aspect, a fitness device is proposed which comprises a frame assembly defining a front side and a back side and including a frame structure which has a bottom part for placing the fitness device on a floor. The fitness device further comprises a mirror fixed to the frame structure which reflects incident light from a user facing a front surface of the mirror to present a reflected image of the user and which transmits incident light from a rear surface of the mirror. At least one display is provided fixed to the frame structure and arranged behind the mirror for displaying exercise content superimposed on at least a portion of the reflected image of the user. In addition, the fitness the device comprises (a) a stand including an attachment portion attached to the back side of the frame assembly above the bottom part of the frame structure and (b) a base portion to be placed on the floor in a distance to the bottom part of the frame structure. The stand extends from the attachment portion to the base portion at an angle to the back side so that the stand includes a force transmitting structure extending along a plane oriented at a specific angle with respect to the back side. By providing a stand which is at least partially angled way from the back side of the frame assembly the fitness device may be self-standing only requiring a small floor area. Naturally, the fitness device may also be hung to a wall (for example, with the stand being removed or folded towards the back side of the frame assembly). By providing an additional inclined stand supporting the frame structure when resting on a floor the installation of the fitness device may be significantly facilitated and the comfort for a customer may be increased.

For example, the front side of the frame assembly and that thus the front side of the fitness device may extend along a plane inclined to the vertical axis in order to provide a favourable viewing angle on a screen of the at least one the display for the user. The stand being angled away from the (inclined) back side provides for a stable support of the frame structure without any risk for a user that the fitness device tilts. The frame structure of the fitness device is rather held in a stable inclined position.

In an exemplary embodiment, the angle at which the stand extends to the back side of the frame assembly is less than 60°, for example less than or equal to 45°. In one embodiment the angle is less than or equal to 25°, thereby allowing for a stable positioning of the fitness device on a floor without the base portion of the stand being positioned in a significant distance to the bottom part of the frame structure. For example, the distance between the bottom part of the frame structure and the base portion of the stand both resting on the floor may be less than one-third of a total height of the fitness device. Compared to the height and thus the overall dimensions of the fitness device the distance between the bottom part and the base portion may be rather small.

In an exemplary embodiment, the attachment portion of the stand is attached to the back side above the bottom part at a mounting region of the back side. The mounting region is located in an upper half of the back side. This may for example include that the back side has a height (measured from the bottom part to a top side of the frame structure) and the mounting region of the back side is located at a distance to the bottom part which is less than two-thirds of the height. In such an embodiment the stand may be thus for example be attached to the back side in an upper half but below an upper third of the back side.

The fitness device may comprise an interface region including at least one of a connector for power supply, a connector for a wired network connection, a connector for a video or sound interface, like an HDMI plug or the like, and a switch, in particular a power switch. The interface region thus for example allows for connecting the fitness device to a power supply, at least one computing device and/or a network via at least one cable. The interface region may for example be disposed at the back side of the frame structure so that the interface region is easily accessible at the back side. In an exemplary embodiment the attachment portion of the stand is attached to the back side above the interface region. Thereby, the interface region is still accessible for a person below the attachment portion and may still be at least partially covered by the stand.

In an exemplary embodiment, the stand is U-shaped, triangular-shaped or rectangular-shaped. For example, the stand comprises two parallel lateral bars connected to the base portion. The two parallel lateral bars may extend from the back side at the specific angle towards an end connected by the base portion. The two parallel lateral bars may include at least a part of the attachment portion of the stand and/or may be connected to a component of the stand including the attachment portion. In an exemplary embodiment, the two parallel lateral bars are connected to a crossbar of the stand which is fixed to the back side. Such a crossbar may be attached at a mounting region of the back side and also connect the two parallel lateral bars so that the crossbar defines at least a part of the attachment portion of the stand and provides for further stiffness of the stand.

Alternatively or additionally, each one of the two parallel lateral bars may be fixed to the back side. In such an embodiment each lateral bar may be separately fixed to the back side, for example via at least one fastening element, such as a screw, a bolt or a rivet, whereas an additional fixation of the stand to the frame assembly is provided via a crossbar of the stand fixed at the back side, for example via at least one additional fastening element. For example, the two parallel lateral bars may be provided with a separate rigid connection to the frame structure and at the back side, whereas the stand is additionally fixed via the crossbar at a mounting region of the back side.

For facilitating assembly of the fitness device and for facilitating arrangement of the stand properly at the specified angle relative to the back side during assembly, the attachment portion of the stand may comprise at least one inclined mounting surface (inclined with respect to a direction of extension of the stand from its attachment portion to its base portion). The at least one inclined mounting surface abuts against the back side when the fitness device is assembled.

The frame structure may define a continuous a frame inside which the mirror is held. In one embodiment, the mirror abuts on the bottom part and a top part of the frame as well as on two opposing side members connecting the bottom part and the top part. A Mirror whose front surface extends over substantially the whole front side (except for the part of the frame structure at the circumference of the front side) is thus securely held within a frame of the frame structure which protects the mirror edges from damages and also facilitates assembly of the fitness device. In an embodiment the frame of the frame structure has a thickness of less than 3 mm or even less than 2 mm. At the circumference of the mirror the frame is therefore rather slim and almost not perceivable at the front side.

In an exemplary embodiment, the fitness device further comprises a back plate at the back side which is attached to the frame structure. The stand may be mounted to the back plate. The back plate may constitute a protective cover at the back side of the fitness device and may for example also include the above-mentioned interface region.

According to a further aspect, a proposed solution fitness device comprises a frame assembly defining a front side and a back side and including a frame structure with a bottom part for placing the fitness device on a floor. Furthermore, the fitness device comprises at least one display fixed to the frame structure for displaying exercise content to a user facing the front side. A stand is part of the fitness device which includes an attachment portion attached to the back side of the frame assembly above the bottom part of the frame structure and which included a base portion to be placed on the floor in a certain distance to the bottom part of the frame structure. The stand may extend from the attachment portion to the base portion at an angle to the back side.

In an exemplary embodiment, the fitness device may additionally comprise a mirror or a protective glass cover fixed to the frame structure, wherein the at least one display is arranged behind the mirror or the protective glass cover. In case of a mirror being part of the fitness device the mirror may reflect incident light from the user facing a front surface of the mirror to present a reflected image of the user and may transmit incident light from a rear surface of the mirror. In such an embodiment the at least one display may thus be arranged behind the mirror for displaying the exercise content superimposed on at least a portion of the reflected image of the user.

According to another aspect of the proposed solution, a fitness device comprises a frame assembly defining a front side and a back side and including a frame structure with a bottom part for placing the fitness device on a floor. Further, the fitness devices may comprises at least one display for displaying exercise content to a user facing the front side. The at least one display is held at the frame structure in a display area of the frame assembly. Additionally, at least one camera is provided for capturing the user while displaying exercise content via the at least one display. The fitness device further comprises a stand including an attachment portion attached to the back side of the frame assembly above the bottom part of the frame structure and including a base portion to be placed on the floor, wherein the attachment portion is attached to the back side at or proximate to the display area of the frame assembly. The at least one camera may be coupled to electronics and in particular an electronic feedback unit of the fitness device configured to provide feedback to the user performing an exercise in the front of the fitness device while exercise content is displayed via the at least one display. Attaching the stand at or proximate to the display area of the frame assembly makes sure that the stand provides sufficient support and stability to the frame assembly at a region where most of the weight of the fitness device is concentrated (assuming that the at least one display makes up for the majority of a total weight of the fitness device).

In an exemplary embodiment, the front side of the fitness device has a height and a width and the at least one display has a display height which is greater than one half of the height of the front side and has a display width which is greater than one half of the width of the front side. Accordingly, the at least one display of the proposed stand-alone fitness device may be relatively large and may provide, for example, for a screen having a size of more than 34 inches, in particular more than 38 inches or 40 inches.

It its furthermore understood that also fitness devices according to the second and third aspects may include several or all features of embodiments disclosed before and in the following with respect to the 1st aspect.

### BRIEF DESCRIPTION OF THE DRAWINGS

FIG. 1A is a perspective view on an embodiment of a proposed fitness device with a view to a front side;

FIG. 1B is a perspective view of the fitness device with a view to a back side;

FIG. 2A is a front view of the fitness device;

FIG. 2B is back view of the fitness device;

FIG. 3 is a side view of the fitness device illustrating dimensions of and angles between components of the fitness device;

FIG. 4 is exploded view of the fitness device;

FIG. 5 is an enlarged detail of the back side of the fitness device showing a mounting region for a stand of the fitness device; and

FIG. 6 is an enlarged detail of a bottom part of a frame structure of the fitness of device.

### DESCRIPTION OF THE INVENTION

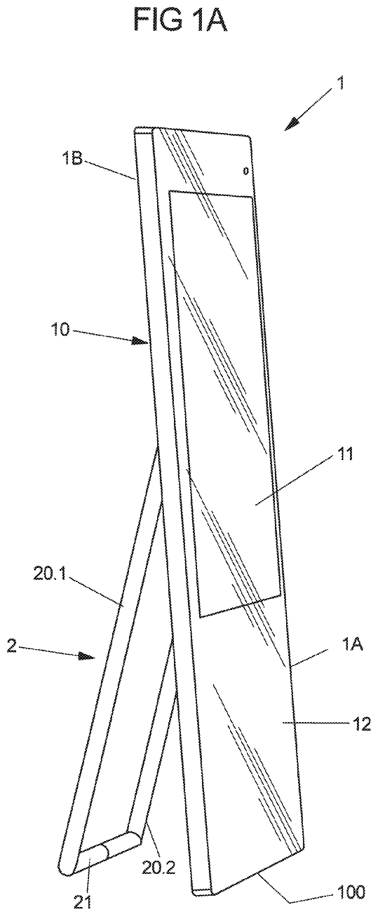

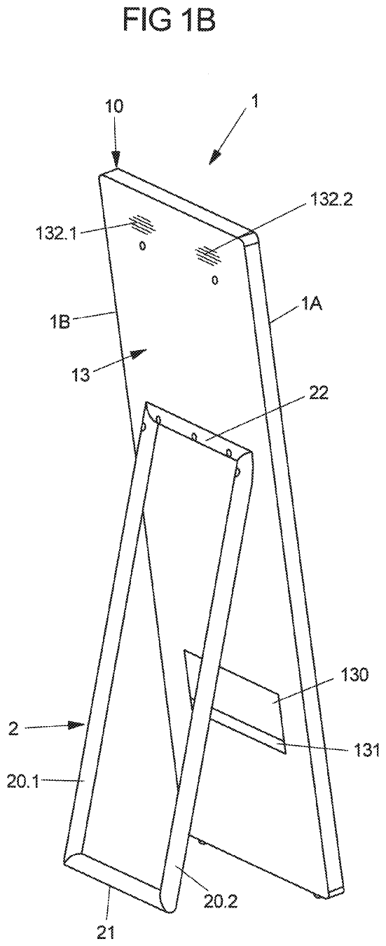

FIG. 1 illustrates an embodiment of a proposed fitness device 1. The fitness device 1 of the embodiment of FIG. 1 is configured as a stand-alone fitness device to be used in a gym or at home. Fitness device 1 comprises a frame assembly including a frame structure 10, a display 11 and a mirror or protective glass cover 12. The frame structure 10 defines a continuous frame surrounding the mirror or protective glass cover 12. The mirror or protective glass cover 12 is thus held within the frame of the frame structure 10. The display 11 is held behind the mirror or protective glass cover 12. The display 11 includes a screen for displaying exercise content to a user standing in front of the fitness device 1. The frame assembly of the fitness device 1 defines a front side 1A and a back side 1B. The exercise content displayed via the display 11 may hence be watched by a user facing the front side 1A.

In case the display 11 is disposed behind a mirror 12 fixed to the frame structure 10 the mirror 12 being may reflect incident light from the user facing a front surface of the mirror 12 to present a reflected image of the user. The mirror 12 may further transmit incident light from the rear surface of the mirror 12 so that exercise content displayed via the display 11 may be superimposed on at least a portion of the reflected image of the user. By superimposing exercise content on at least a portion of the selected image of the user the fitness device 1 may provide instant feedback on a performance of the user when imitating fitness exercises based on the displayed exercise content.

Alternatively or additionally, a camera may be part of the fitness device to capture images of the user during a fitness exercise in order to electronically provide feedback on the user's performance whether the user imitates a certain exercise properly and, e.g., to which extent.

The fitness device 1 as shown in FIG. 1A includes the display 11 having a screen with a diameter of at least 34 inches, for example more than 40 inches. Accordingly, the frame structure 10 defines a surface area at the front side 1A of more than 0.5 m². For example, a substantially rectangular surface at the front side 1 measures more than 1.5 m in height and more than 0.4 m in width.

Given the size and weight of the fitness device 1 a secure and stable position of the fitness device 1 with its frame structure 10 is crucial. For this reason the fitness device 1 of FIG. 1A may rest via at least two different components at a floor. A first component is provided by bottom part 100 of the frame structure 10. The bottom part 100 allows the frame structure 10 to rest on the floor and thus provides a support directly below the display 11. In addition, a stand 2 is provided which allows the frame structure 10 to be positioned inclined to the vertical at the floor and to be nevertheless held in a stable position. The stand 2 extends at an angle to the back side 1B of the frame structure 10 so that a base portion of the stand 2 (formed by a base member 21) is configured to rest on the floor being the bottom part 100 in a specified distance to the bottom part 100.

As further illustrated by FIG. 1B, the stand 2 of this embodiment is designed as a rectangular frame including a base member 21, a crossbar and 22 two parallel lateral bars 20.1, 20.2. The lateral bars 20.1, 20.2 are connected to each other at a first (upper) end via the crossbar 22 and at a second (lower) end via the base member 21. At the crossbar 22 the stand 2 is fixed to a back plate 13 the fitness device 1. The back plate 13 is attached to the frame structure 10 at the back side 1B.

In a display area of the frame structure 10 at which at the display 11 is held, the mounting plate 13 defines a mounting region. At this mounting region the crossbar 22 is attached to the back plate 13 via an attachment portion of the stand 2. The stand 2 is fixed to the back plate 13 (and for example a component of the frame structure 10 covered by the back plate 13) via several fastening elements, for example in the form of screws, bolts or rivets which at least partially extend through the attachment portion at the crossbar 22.

The attachment portion of the crossbar 22 is fixed to the back plate 13 at a location above a recess portion or recess 130 of the back plate 13 and below two grille portions 132.1 and 132.2 for loudspeakers of the fitness device 1. The recess 130 includes an interface region 131. At an edge section of the recess 130 the interface region may accessible for the user and may comprise at least one of a connector for a power supply, a connector for a wired network connection, one or more connectors for a video or sound interface and a main power switch.

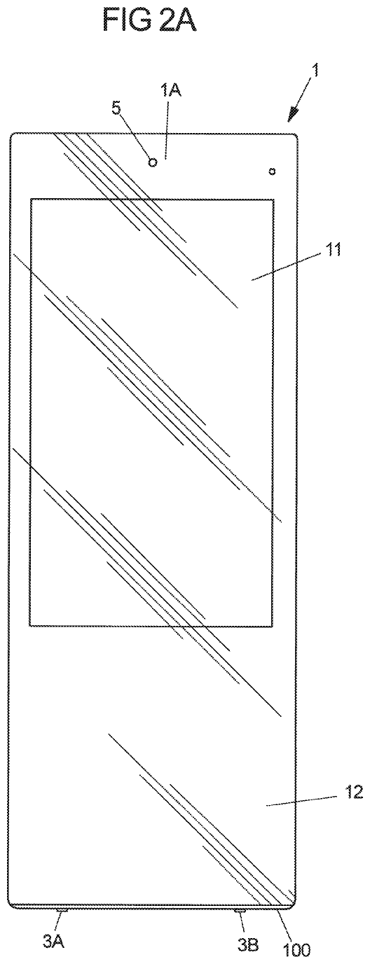

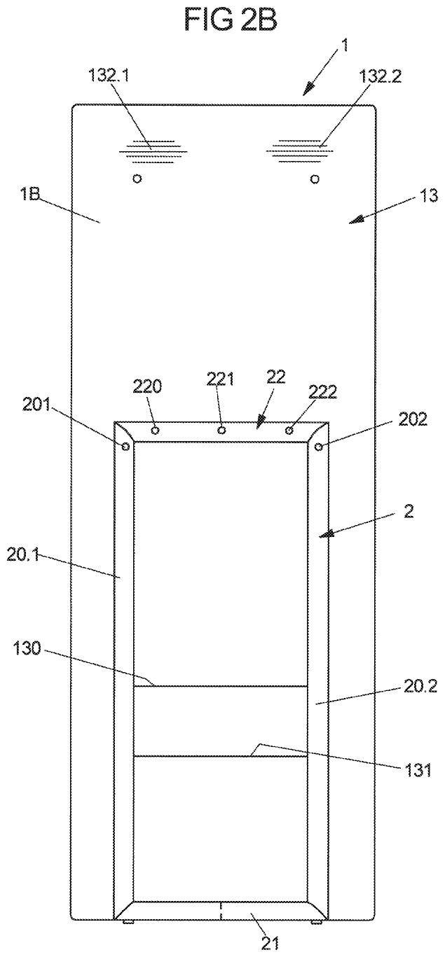

As can be seen from the front view of FIG. 2A, the fitness device 1 may for example in particular include one or more cameras 5 for capturing images of the user facing the front side 1A. For example, a camera 5 may be positioned at the front side 1A in the middle of a portion above the display 11.

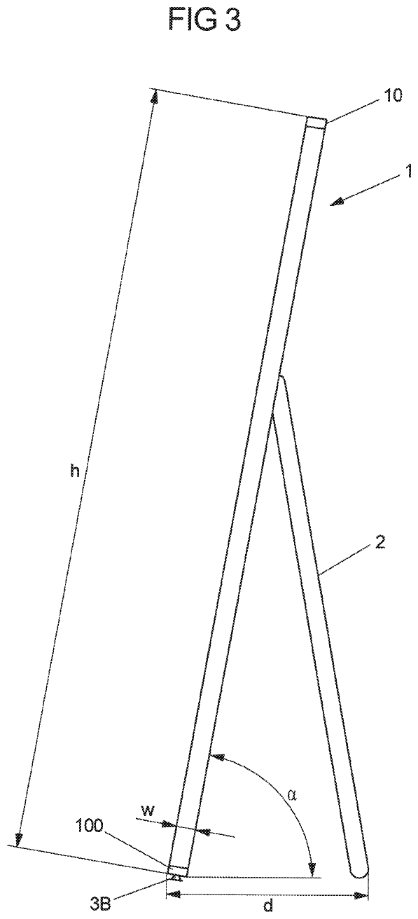

The back view of FIG. 2B further illustrates the position, dimensions and the configuration of the stand 2 with respect to the frame structure 10. Here, a height of the stand 2 is greater than one half of a total height h of the frame structure 10 (cf. also FIG. 3). Thereby, the attachment portion of the crossbar 22 is fastened to the mounting region at the back side 1A at a height (distance to the bottom part 100) which is greater than one half of the total height h of the frame structure 10 but less than two-thirds of the total height.

In the shown example, the crossbar 22 is fixed to the back plate 13 via three fastening elements, for example three screws, at three mounting holes 220, 221, and 222. In addition, the first (upper) end of each lateral bar 20.1, 22.2 is separately fixed to the back plate 13 via mounting holes 201, 202 for providing an additional fixation of the stand 2 and its parts to the back plate 13 and thus to the frame assembly of the fitness device 1.

As can be seen from the side view of FIG. 3, the frame of the frame structure 10 has a total height h which is several times bigger than a width w of the frame. Accordingly, the frame of the frame structure 10 is relatively thin or slim which provides for an elegant outer appearance of the fitness device 1. For example, the width w lies in the range of 2 mm to 5 mm, whereas the total height h is the greater of then 1.5 m, for example lies in the range of 1.5 m to 1.8 m.

The stand 2 extends from its attachment portion at the crossbar 22 to its base member 21 at an angle of about 18° to 25° to the frame structure 10. Thereby, the frame structure 10 (and the display 11) may be held in a stable inclined position. Accordingly, the frame structure 10 does not extend in a completely vertical plane but with a certain inclination to the vertical, namely at an angle α of less than 90° relative to the floor. For example, the angle α lies in the range of 75° to 82°.

The base member 21 of the standard 2 is furthermore resting on the floor at a distance d to the bottom part 100. For example, the distance d lies in the range of 40 cm to 50 cm.

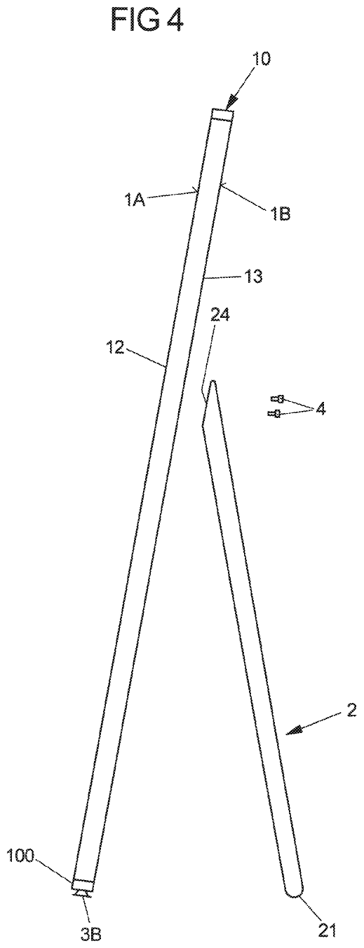

The exploded view of FIG. 4 in particular illustrates an inclined mounting interface of the stand 2 for abutting on the back side 13. Via the inclined mounting interface 22 the stand 2 may abut a long defined plane area against the back plate 30 while the stand 2 being angled away from the back plate 13. FIG. 4 furthermore illustrates screws 4 to be used for fastening the stand 2 via the mounting holes 220, 221, 222 and 201, 202 to the back plate 30 and also to a part of the frame structure 10 covered by the back plate 13.

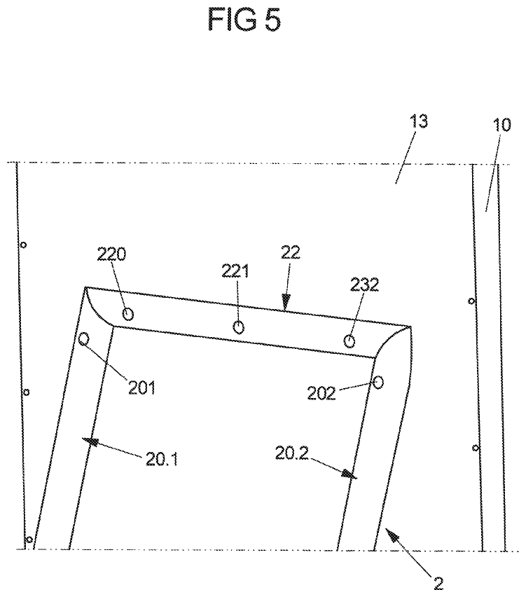

The enlarged view of FIG. 5 shows that the mounting holes 220, 221 and 222 of the cross bar 22 as well as the mounting holes 201 and 202 of the two lateral bars 20.1 and 20.2 in greater detail.

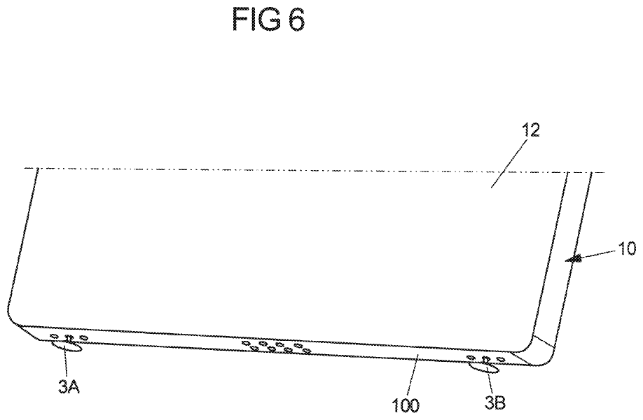

The enlarged view of FIG. 6 furthermore illustrates that the bottom part 100 of the frame structure 10 may include at least two height adjusters 3A, 3B. Via this height adjusters 3A, 3B of the bottom part 100 the frame structure 10 may rest on the floor. By turning the height adjusters 3A, 3B in a threaded hole of the frame structure 10 irregularities of the floor may be compensated for and an inclination of the frame structure may be adjusted.

The foregoing description of the embodiments has been provided for purposes of illustration and description. It is not intended to limit the disclosure. Individual elements or features of a particular embodiment are generally not limited to that particular embodiment, but, where applicable, are interchangeable and can be implemented in another embodiment, even if not specifically shown or described. The same elements may also be varied in one or more ways. Such variations are not to be regarded as a departure from the disclosure, and all such modifications are intended to be included within the scope of the disclosure.

What is claimed is:

1. A fitness device, comprising:
a frame assembly defining a front side and a back side and including a frame structure which has a bottom part for placing the fitness device on a floor;
a mirror fixed to the frame structure which reflects incident light from a user facing a front surface of the mirror to present a reflected image of the user, and which transmits incident light from a rear surface of the mirror;
at least one display fixed to the frame structure and arranged behind the mirror for displaying exercise content superimposed on at least a portion of the reflected image of the user; and
a stand including an attachment portion attached to the back side of the frame assembly above the bottom part of the frame structure and a base portion to be placed on the floor in a distance to the bottom part of the frame structure,
wherein the stand extends from the attachment portion to the base portion at an angle to the back side.

2. The fitness device of claim 1, wherein the angle at which the stand extends to the back side is less than 60 degrees.

3. The fitness device of claim 2, wherein the angle at which the stand extends to the back side is less than or equal to 45 degrees.

4. The fitness device of claim 3, wherein the angle at which the stand extends to the back side is less than or equal to 25 degrees.

5. The fitness device of claim 1, wherein the attachment portion of the stand is attached to the back side above the bottom part at a mounting region of the back side which is located in an upper half of the back side.

6. The fitness device of claim 5, wherein the back side extends with a height from the bottom part to a top side of the frame structure and the mounting region of the back side is located at a distance to the bottom part which is less than two-thirds of the height.

7. The fitness device of claim 1, wherein the fitness device comprises an interface region including at least one of a connector for power supply, a connector for a wired network connection, a connector for a video or sound interface and a switch.

8. The fitness device of claim 7, wherein the interface region is disposed at the back side and the attachment portion of the stand is attached to the back side above the interface region.

9. The fitness device of claim 1, wherein the stand is U-shaped, triangular-shaped or rectangular-shaped.

10. The fitness device of claim 1, wherein the stand comprises two parallel lateral bars connected by the base portion.

11. The fitness device of claim 10, wherein the two parallel lateral bars are connected to a crossbar fixed to the back side.

12. The fitness device of claim 10, wherein each one of two parallel lateral bars is fixed to the back side.

13. The fitness device of claim 1, wherein the attachment portion of the stand comprises at least one angled mounting surface abutting against the back side.

14. The fitness device of claim 1, wherein the frame structure defines a continuous frame inside which the mirror is held.

15. The fitness device of claim 1, comprising a back plate at the back side being attached to the frame structure to which back plate the stand is mounted.

16. A fitness device, comprising:
a frame assembly defining a front side and a back side and including a frame structure which has a bottom part for placing the fitness device on a floor;
at least one display fixed to the frame structure for displaying exercise content to a user facing the front side; and
a stand including an attachment portion attached to the back side of the frame assembly above the bottom part of the frame structure and a base portion to be placed on the floor in a distance to the bottom part of the frame structure,
wherein the stand extends from the attachment portion to the base portion at an angle to the back side.

17. The fitness device of claim 16, comprising a mirror fixed to the frame structure which reflects incident light from the user facing a front surface of the mirror to present a reflected image of the user, and which transmits incident light from a rear surface of the mirror,
wherein the at least one display is arranged behind the mirror for displaying the exercise content superimposed on at least a portion of the reflected image of the user.

18. The fitness device of claim 16, comprising a protective glass cover fixed to the frame structure, wherein the at least one display is arranged behind the protective glass cover.

19. A fitness device, comprising:
a frame assembly defining a front side and a back side and including a frame structure which has a bottom part for placing the fitness device on a floor;
at least one display for displaying exercise content to a user facing the front side, wherein the at least one display is held at the frame structure in a display area of the frame assembly;
at least one camera for capturing the user while displaying the exercise content via the least one display; and
a stand including an attachment portion attached to the back side of the frame assembly above the bottom part of the frame structure and including a base portion to be placed on the floor,
wherein the attachment portion is attached to the back side at or proximate to the display area of the frame assembly.

20. The fitness device of claim 19, wherein the front side has a height and a width and the at least one display has a display height which is greater than a half of the height of the front side and has a display width which is greater than a half of the width of the front side.
